(19) United States
(12) Patent Application Publication
(10) Pub. No.: US 2012/0184866 A1
(43) Pub. Date: Jul. 19, 2012

(54) IMPLANTABLE MEDICAL DEVICE AND METHOD FOR MULTISITE MEASUREMENT OF INTRACARDIAC IMPEDANCE

(75) Inventors: Nils Holmstrom, Jarfalla (SE); Anders Bjorling, Solna (SE)

(73) Assignee: St. Jude Medical AB, Jarfalla (SE)

(21) Appl. No.: 13/498,568

(22) PCT Filed: Sep. 29, 2009
(86) PCT No.: PCT/SE2009/000428

§ 371 Date: Mar. 27, 2012

(51) Int. Cl. A61B 5/0408

(52) U.S. Cl. 600/509

(57) ABSTRACT

The present invention generally relates to an implantable medical device and method for detecting and monitoring cardiac status of a patient using simultaneous multisite measurements of the intracardiac impedance and in particular to ischemia detection using the simultaneous multisite measurements. The device comprises an impedance measuring unit being connectable to a plurality of electrode configurations including a current generating device adapted to generate a current and apply the current between two electrodes of a current injecting electrode configuration of the electrode configurations and a voltage sensing device including a plurality of voltage sensing circuits arranged in parallel. Each voltage sensing circuit being connectable to a specific voltage sensing electrode configuration of the electrode configurations and being arranged to sense a voltage over the voltage sensing electrode configuration resulting from the applied current, wherein the voltage sensing circuits are capable of sensing the resulting voltages simultaneously. The device further comprises an impedance calculating module adapted to calculate a plurality of impedance values, each impedance value being based on the applied current and a resulting voltage.

### TECHNICAL FIELD

The present invention generally relates to implantable medical devices, such as pacemakers, and, in particular, to techniques for detecting and monitoring cardiac status of a patient using simultaneous multisite measurements of intracardiac impedance.

### BACKGROUND OF THE INVENTION

Improved and more accurate measurements of e.g. intracardiac impedance may entail many advantages in an implantable medical device such as a pacemaker.

For example, heart failure (HF) is a common diagnosis and entails enormous costs for the society in terms of money and suffering due to drug costs, hospitalization, and pain for the patient. There are two groups of HF, ischemic and non-ischemic cardiomyopathy. The non-ischemic disorders can be divided in dilated cardiomyopathy, hypertrophic cardiomyopathy, and restrictive cardiomyopathy.

There exists a need of improved methods and devices for monitoring HF status of patients and for detecting HF progression.

Due to the in general poorer medical status of pacemaker and ICD patients they are subjected to an increased risk of ischemic heart disease (IHD) and myocardial infarction (MI). At sudden ischemic events e.g. myocardial infarction, the risk for serious arrhythmia is high or elevated. Further, ischemic heart disease can be divided into unstable and stable ischemia. Unstable ischemia is a highly life-threatening situation most often caused by an infarction of one or several coronary arteries. Sudden cardiac death depends in 90% of all cases on IHD. The possibility to survive depends on how fast the patient gets relevant treatment. Stable ischemia is very common among elderly people and the mortality is low or moderate. There is both silent ischemia and ischemia with heart pain, i.e. angina pectoris. The most common reason is arteriosclerosis in the coronary vessels. It can be treated with drugs, operatively using bypass techniques, stents, etc. When IHD progresses it may lead to myocardial infarction (MI), congestive heart failure (CHF) and/or the patient's death. In fact early detection of IHD can serve as an early marker for CHF risk factor. An early detection of ischemic heart disease is thus required since that will give opportunities to prevent life threatening complications.

Thus, there exists a need within the art of methods and device capable of detecting the occurrence of an ischemic episode and of determining the location of the ischemia with high accuracy and reliability. If intracardiac impedance values are obtained sequentially, hemodynamic changes due to e.g. posture, respiration or workload may occur between the sequentially performed measurements, there is a significant risk that signals obtained under different conditions are compared during the detection. If a part or a region of a heart is subjected to ischemia, this part or region will behave differently compared to the behaviour before the onset of the ischemia or in comparison to other non-ischemic parts or regions. Both the contraction and the relaxation of the myocytes will be slower during the ischemia. However, the hemodynamic behaviour can also change due to other circumstances such as, for example, a changed workload, change of body posture, medication, preload and afterload. Under such circumstances the myocardium will be affected globally and not regionally as in the case of an ischemic event. Hence, it is of a great importance in ischemia detection to be able to discriminate between a true ischemic event and a hemodynamic or metabolic change, which may be very difficult if the impedances are measured sequentially. Also, measuring different vectors sequentially may require averaging the signal to avoid beat-to-beat differences. This averaging should be made over several respiration cycles to eliminate the influence of such beat-to-beat differences. Hence, the measurement time for every vector may require several seconds and accordingly, if several vectors are studied, the total measurement time for obtaining an impedance pattern may require minutes. Ischemic events requires immediate response actions by e.g. alarming or change of therapy, which means that is a matter of a few minutes that may be the difference between life and death of the patient.

Accordingly, there is a need of an improved method and device for detecting and locating an ischemic episode and for improving the specificity in such detection of ischemic episodes.

The incidence of inappropriate shocks in patients with an implanted cardioverter defibrillator (ICD) is, despite significant improvements during recent years, still far too high. Such inappropriate shocks cause unnecessary suffering to the patients. Therefore, it is of high importance to improve the detection methods further to avoid these unnecessary or inappropriate shocks. To be able to distinguish between stable and unstable tachycardia is an important area of improvement for today's IDC therapy. Accordingly, there is a need for improved methods and device that are capable of distinguishing between stable and unstable tachycardia.

Moreover, HF patients have a reduced ability to compensate for defective timings of the heart contraction pattern. Accordingly, it is especially important to optimize the atrioventricular and interventricular pacing intervals in these patients. However, also patients without HF may develop HF by time if the pacemaker therapy is suboptimal.

### SUMMARY OF THE INVENTION

The present invention provides an improved medical device and method that are capable of fulfilling at least some of the above-mentioned needs or provide a solution to or alleviating at least some of the above-mentioned problems in the prior art.

An object of the present invention is to provide an improved medical device and method that are capable of simultaneous multisite measurements of intracardiac impedance.

This and other objects of the present invention are achieved by means of a method and an implantable medical device having the features defined in the independent claims. Different embodiments of the invention are characterized by the dependent claims.

In the context of the present application, the term “impedance” refers to complex impedance consisting of the resistance (the real part) and the reactance (the imaginary part) and thus the term “impedance value” may, for example, refer to a resistance value and/or a reactance value, as well as a phase angle of the impedance or an absolute value of the impedance, and, additionally, or the admittance. Further, the impedance value may also be, for example, the time derivative of the resistance and/or the reactance, or the phase angle.

In the present application, the term “impedance pattern” refers to the pattern or map of different impedance values obtained by the different electrode pairs during a multisite measurement reflecting the tissue response at the different electrode pair sites. Since the measurements are performed simultaneously at a number of different tissue sites, the impedance pattern or impedance map over the heart illustrates the impedance at the different tissue sites in one single beat.

According to a first aspect of the present invention, there is provided an implantable medical device connectable to at least one medical lead including a plurality of electrodes for contact with tissue of a heart of a patient. The device comprises an impedance measuring unit being connectable to a plurality of electrode configurations including a current generating device adapted to generate a current and apply the current between two electrodes of a current injecting electrode configuration of the electrode configurations and a voltage sensing device including a plurality of voltage sensing circuits arranged in parallel. Each voltage sensing circuit being connectable to a specific voltage sensing electrode configuration of the electrode configurations and being arranged to sense a voltage over the voltage sensing electrode configuration resulting from the applied current, wherein the voltage sensing circuits are capable of sensing the resulting voltages simultaneously. The device further comprises an impedance calculating module adapted to calculate a plurality of impedance values, each impedance value being based on the applied current and a resulting voltage.

According to a second aspect of the present invention, there is provided a method for an implantable medical device connectable to at least one medical lead including a plurality of electrodes for contact with tissue of a heart of a patient and including an impedance measuring unit being connectable to a plurality of electrode configurations. The method comprises: generating a current and apply the current between two electrodes of a current injecting electrode configuration of the electrode configurations, sensing voltages at a plurality of voltage sensing circuits arranged in parallel, each voltage being sensed over specific voltage sensing electrode configuration resulting from the applied current, wherein the resulting voltages are sensed simultaneously, and calculating a plurality of impedance values, each impedance value being based on the applied current and a resulting voltage.

In the prior art it is possible to obtain an image over the heart to be used in e.g. ischemia detection and determination of the location or area in the heart of an ischemic event using, for example, so called impedance tomography. To this end, intracardiac or cardiogenic impedance is consecutively measured over several measurement vectors, i.e. at one electrode configuration at the time. Thereby, an impedance image over the heart can be obtained. The different voltage responses from different electrode configurations or combinations will mirror conduction patterns, volume changes etc. from different parts or areas of the heart. Depending on the site of the current induction, i.e. the location of the current injection electrodes, and of the voltage sensing electrodes, i.e. the locations of the different electrode pairs, the response will be different.

However, the different impedance values will not be synchronized in time, which may induce, for example, respiration artifacts rendering the impedance image less accurate. Therefore, in order to reduce, for example, respiration artifacts, when comparing or using impedance signals from several different electrode configurations, average waveforms from several heartbeats are created for each electrode configuration.

The present invention is, on the other hand, based on the idea of measuring each impedance value, at the different electrode configurations, simultaneously. This can be achieved due to the design of the impedance measurement unit according to the present invention comprising a current generating device adapted to generate a current and to apply the current between two electrodes and a voltage sensing device including a plurality of voltage sensing circuits arranged in parallel. Each voltage sensing circuit is connected to an electrode pair and is adapted to sense a voltage resulting from the applied current at the electrodes. Thereby, the analysis of the plurality of measurement vectors can be made on beat-to-beat basis. Variations due to the respiration can be accepted since the same influence will be seen in each impedance signal due to the simultaneous recording. In fact, the variations due to the respiration can even be utilized to improve the sensitivity and specificity to detect variation caused by, for example, heart failure or ischemic episodes. Especially, sudden events can be detected much more rapid or swift in comparison to the sequential approach used in the prior art, which, as discussed above, may be of a great importance or even crucial for the survival of the patient.

Moreover, heart failure monitoring and detection can be made more accurate and reliable. As a patient's heart failure status improves or worsens, the heart chamber's sizes changes. As an example, when a patient's heart failure worsens, the LA volume can increase while the LV volume remains fairly constant. However, in case of restrictive cardiomyopathy, the LV volume will actually decrease since the heart muscle will grow. Thus, by measuring the volume of the LV as well as of the LA using impedance, simultaneously at a number of different sites, a very accurate heart failure progression can be obtained but also the type of heart failure can be determined. Furthermore, since the impedance is affected by the thickness of the myocardial tissue, the simultaneous multisite measurements of the impedance will give a picture of the tissue thickness at the different measurement sites and thus the detection and characterization of heart failure can be further improved.

Furthermore, the arrhythmia discrimination can also be made more accurate and reliable using the present invention. If the impedance in the atrium and the impedance in the ventricle are measured simultaneously, the synchronicity between the chambers can be determined with a high degree of accuracy. The AV synchronicity is important in determining whether a detected fast ventricular rhythm originates from the ventricle or from the atrium.

An optimization of the device parameter settings can be made more accurate and fast using the present invention. For example, typical parameters that can be optimized are the AV delay and the W delay. Heart failure patients are sensitive to the excessively high stimulation rate at e.g. increased activity. Over-pacing and also too low pacing rate results in abnormal contraction patterns which can be detected by the simultaneous multisite impedance measurements. Analyses of the relation between the detector signals at different parameter settings give information about the change of cardiac contraction pattern, which can be of great value during, for example, continuous optimization of the heart stimulation. The optimal stimulation parameter setting can be identified and determined using echo or other external equipment, for example, at a set-up session at the health-care facility. An impedance pattern measured at a specific parameter setting or specific settings are stored as reference values. Thereafter, during the daily operation, the stimulation parameter settings may be continuously adjusted to obtain the same or a similar impedance pattern as the reference pattern.

Sudden ischemia or progression of existent ischemia can also be detected with an improved accuracy and reliability by using the present invention. By using the impedance pattern obtained by the simultaneous multisite measurements of the impedance, i.e. the tissue response from different areas of the heart muscle depending on the configuration of electrode pairs, together with timing information (i.e. how the impedance values are related to different cardiac events such as, for example, the R-wave or the T-wave), it is possible to detect changes and mechanical contraction as well as the location where the change is observed. The timing information can be provided by synchronizing IEGM data with the impedance measurements. Hence, it is possible to alarm for sudden ischemic events and indicate the location of the ischemic event. It is also conceivable to initiate preventive therapy to avoid arrhythmic complications.

An object of an embodiment of present invention is to provide implantable medical device including an ischemia detector capable of detecting an onset of ischemia at an early stage and to detect a location of ischemia with a high degree of accuracy. According to an embodiment of the present invention, an ischemia detector evaluates the impedance values to detect changes in the impedance values being consistent with an ischemia and to detect a location of the ischemia. In particular, the impedance values are compared with a reference impedance matrix or pattern. If a part or a region of a heart is subjected to ischemia, this part or region will behave differently compared to the behaviour before the onset of the ischemia or in comparison to other non-ischemic parts or regions. Both the contraction and the relaxation of the myocytes will be slower during the ischemia. However, the hemodynamic behaviour can also change due to other circumstances such as, for example, a changed workload, change of body posture, medication, preload and afterload. Under such circumstances the myocardium will be affected globally and not regionally as in the case of an ischemic event. Therefore, the specificity of the ischemia detection can be improved by measuring the beat-to-beat response at several different locations of the heart simultaneously. If the impedance values are gathered simultaneously during the same contraction at several areas of the heart (i.e. by means of several measurement vectors) and a change is observed in only a limited part of the heart (i.e. in one or only in few impedance values), it is therefore a high probability that an ischemic event is identified. Further, the location can also be identified since each impedance value is associated with a specific measurement vector, which in turn, measures the impedance in an area or a region of the heart. Thus, by localizing which vector(-s) that differ from a baseline (a reference) or from the other vectors an indication of which artery is subjected to coronary insufficiency or ischemia. On the other hand, if the change is seen in all impedance values (i.e. in all measurement vectors) it is more likely a global hemodynamic or metabolic change that not is a result of a local occlusion of a coronary artery.

If the impedance measurements over different vectors are measured sequentially, hemodynamic changes due to e.g. posture, respiration or workload may occur between the measurements and the risk that signals obtained under different conditions are compared will be high. In ischemia detection it is important, as described above, to discriminate between a true ischemic event and a hemodynamic or metabolic change, which will be very difficult if the impedances are measured sequentially. Also, measuring different vectors sequentially may require averaging the signal to avoid beat-to-beat differences. This averaging should be made over several respiration cycles to eliminate the influence of such beat-to-beat differences. Hence, the measurement time for every vector may require several seconds and accordingly, if several vectors are studied, the total measurement time for obtaining an impedance pattern may require minutes. Ischemic events requires immediate response actions by e.g. alarming or change of therapy, which means that is a matter of a few minutes that may be the difference between life and death of the patient. By measuring all vectors simultaneously, response actions can be taken in principle directly or at least much earlier in comparison to the sequential case used in the prior art.

Furthermore, with continuous, simultaneous multi-vector measurement of impedance it is possible to study details of the impedance waveforms from different part or regions of the heart at exactly the same time. For example, the period of time from the R-wave to the maximum impedance slope. This period of time for each measurement vector can be compared to the corresponding vectors at baseline (i.e. during healthy conditions) to identify sudden increases or decreases in one or several of these periods of time. The studied details of the impedance waveform of one single heart beat can also be compared to the corresponding detail of consecutive heart beats to create trends over time.

Moreover, the signal to noise ratio (SNR) can be improved significantly by using the present invention. For example, by measuring the voltage between pairs of electrodes that are either very close to each other or that constitutes measurement vectors that are similar to each other, e.g. RV tip electrode and LV ring electrode, and RV ring electrode and LV tip electrode, similar signals will be obtained since the electrode configurations will reflect similar volume changes, similar tissue characteristics, etc. If these signals are averaged to form an average impedance value, the SNR will be reduced, which, in turn, will improve the accuracy and reliability of the application the signal is used in.

According to embodiments of the present invention, the impedance value is, for example, the resistance and/or the reactance and/or the absolute value of the impedance and/or the phase angle of the impedance. Further, the impedance value may also be the admittance.

In embodiments of the present invention, the time derivative of the impedance is calculated, e.g. the time derivative of the resistance and/or the reactance, and/or the phase angle, and an impedance pattern including the time derivatives is created. By comparing a present impedance pattern with earlier patterns changes of the time derivative of the impedance value over time can be identified, which may indicate the onset of ischemia and the location of the ischemia. Thus, it may be possible to identify the onset of ischemia and the location of the ischemia at an early stage.

Due to the fact that the impedance pattern is created of the simultaneously obtained impedance values, an impedance image characterizing the mechanical activity of the heart at a single heart beat. Hence, a single beat hemodynamic characterization of the heart can be achieved.

According to embodiments of the present invention, changes in the calculated impedance values being consistent with an ischemia is detected by analysing amplitude of the impedance values and/or the duration of the respective impedance values (or the time period a respective impedance value is above a predetermined threshold). A location of the ischemia is determined based on the detected changes. That is, a location can be determined by identifying the vector (or vectors) that was (were) used to obtain the changed value or (values). Due to the fact that it is known over which region or part of the cardiac tissue respective vector measures, it is possible to connect a change of a certain impedance value to a certain region or part of the tissue. For example, it has been observed that a decrease in the amplitude of the impedance values of an impedance waveform and/or an extension of the duration of the waveform is consistent with an ischemia and by identifying which impedance waveforms and (thus identifying vectors) that are subjected for these changes, the ischemia can be detected as well as the location of the ischemia.

Furthermore, it also conceivable to study or analyse the resistivity and/or the reactance of the impedance and/or the phase angle, as well as the time derivative of any one, some or all of these parameters or admittance at different frequencies to detect an ischemic event and the location for the event as described in, for example, WO 2008/105692 by the same applicant entitled “Medical device for detecting ischemia and a method for such a device”, which hereby is incorporated in its entirety or “Myocardial Electrical Impedance Mapping of Ischemic Sheep Hearts and Healing Aneurysms”, Fallert A. Michael, et al., Circulation, Vol. 78, No. 1, January 1993. As the skilled person realizes, steps of the methods according to the present invention, as well as preferred embodiments thereof, are suitable to realize as computer program or as a computer readable medium.

Further objects and advantages of the present invention will be discussed below by means of exemplifying embodiments.

### BRIEF DESCRIPTION OF THE DRAWINGS

Exemplifying embodiments of the invention will be described below with reference to the accompanying drawings, in which:

FIG. 1 is a simplified, partly cutaway view, illustrating an implantable medical device according to the present invention with a set of leads implanted into the heart of a patient;

FIG. 2 is a functional block diagram form of the implantable medical device shown in FIG. 1, illustrating basic circuit elements that provide, for example, pacing stimulation in the heart and for acquiring simultaneous'impedance signals from several electrode configurations according to the present invention;

FIG. 3 is a functional block diagram form of an embodiment of the impedance calculating module shown in FIG. 2;

FIG. 4 is a functional block diagram form of another embodiment of the impedance calculating module shown in FIG. 2;

FIG. 5 is a functional block diagram form of a further embodiment of the impedance calculating module shown in FIG. 2;

FIG. 6a is a schematic diagram showing the IEGM and impedance waveforms obtained simultaneously by means of four measurement vectors during normal conditions; and

FIG. 6b is a schematic diagram showing the IEGM and impedance waveforms obtained simultaneously by means of four measurement vectors at the occurrence of an ischemic event.

### DESCRIPTION OF EXEMPLIFYING EMBODIMENTS

The following is a description of exemplifying embodiments in accordance with the present invention. This description is not to be taken in limiting sense, but is made merely for the purposes of describing the general principles of the invention. It is to be understood that other embodiments may be utilized and structural and logical changes may be made without departing from the scope of the present invention. Thus, even though particular types of implantable medical devices such as heart stimulators will be described, e.g. biventricular pacemakers, the invention is also applicable to other types of cardiac stimulators such as dual chamber stimulators, implantable cardioverter defibrillators (ICDs), etc.

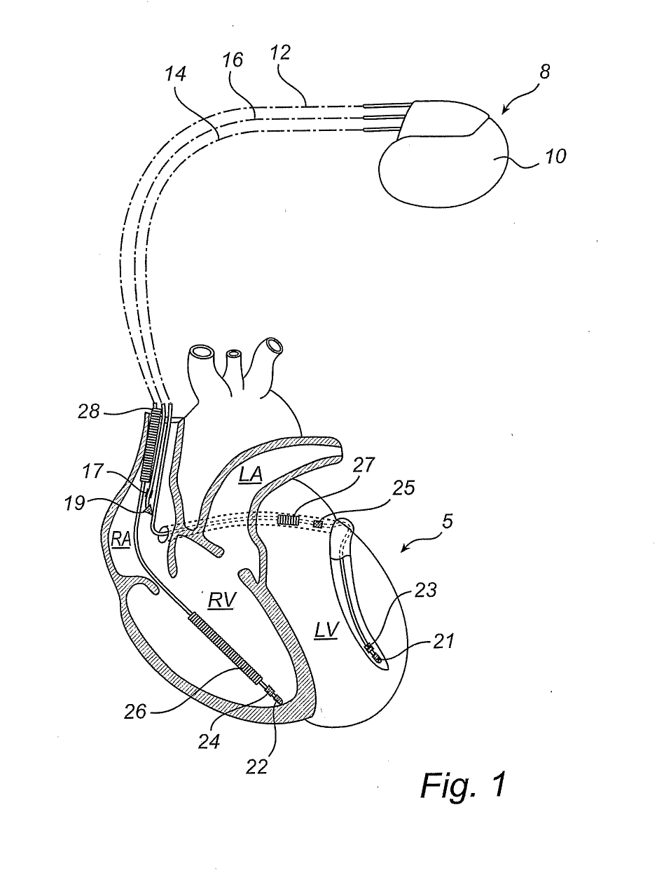

Turning now to FIG. 1, which is a simplified schematic view of one embodiment of an implantable medical device (“IMD”) 8 according to the present invention. IMD 8 has a hermetically sealed and biologically inert case 10. In this embodiment, IMD 8 is a pacemaker which is connectable to pacing and sensing leads 12, 14, in this illustrated case two leads. However, as the skilled person understands, the pacemaker may also be connected to one or several, e.g. three or more, pacing and sensing leads. IMD 8 is in electrical communication with a patient's heart 5 by way of a right ventricular lead 12 having a right ventricular (RV) tip electrode 22, a RV ring electrode 24, RV coil electrode 26, and a superior vena cava (SVC) coil electrode 28. Typically, the RV lead is transvenously inserted into the heart 5 so as to place the RV coil electrode 26 in the right ventricular apex and the SVC coil electrode 28 in the superior vena cava. Accordingly, the right ventricular lead 12 is capable of receiving cardiac signals, and delivering stimulation in the form of pacing to the right ventricle RV.

In order to sense left atrial and ventricular cardiac signals and to provide left chamber pacing therapy, IMD 8 is coupled to a “coronary sinus” lead 14 designed for placement in the coronary sinus region via the coronary sinus for positioning a distal electrode adjacent to the left atrium. As used herein, the wording “coronary sinus region” refers to the vasculature of the left ventricle, including any portion of the coronary sinus, great cardiac vein, left marginal vein, middle cardiac vein, and/or small cardiac vein or any other cardiac vein accessible via the coronary sinus. Accordingly, the coronary sinus lead 14 is designed to receive atrial and ventricular pacing signals and to deliver left ventricular pacing therapy using at least a left ventricular (LV) tip electrode 21, a LV ring electrode 23 left atrial pacing therapy using at least a LA electrode 25 and a LA electrode 27.

Furthermore, a right atrium (RA) lead 16 implanted in the atrial appendage having a RA tip electrode 19 and a RA ring electrode 17 is arranged to provide electrical communication between the right atrium (RA) and the IMD 8.

With this configuration bi-ventricular therapy can be performed. Although three medical leads are shown in FIG. 1, however, it should also be understood that additional stimulation leads (with one or more pacing, sensing, and/or shocking electrodes) may be used.

IMD 8 is an exemplary device that may use the techniques according to the invention. The invention is not limited to the device shown in FIG. 1. For example, while the pacemaker 8 is depicted as a three-chamber pacemaker, the invention can also be practiced in a single-chamber, dual-chamber, or four-chamber pacemaker. According to various embodiments of the present invention, IMD 8 detects electrical cardiac signals, including e.g. the T-wave and the R-wave.

In the embodiment of the present invention illustrated in FIG. 1, a large number of electrode configurations can be used to detect the impedance. For example, the electrical current i(t) may be applied between the RV tip electrode 22 and the LV tip electrode 21. According to the invention, the voltage response u(t)ₙ=v(t)ₘ−v(t)ₙ may be detected between two or more pairs of electrodes simultaneously, for example, between RV coil electrode 26 and LV ring electrode 23, RA tip electrode 19 and RV ring electrode 24, and LV ring electrode 25 and the can 10. However, this is only an arbitrary example, a there are, as the skilled person realizes, a large number of conceivable electrode configurations.

The different voltage responses from the different electrode configurations will mirror conduction patterns, volumes changes, etc. at different parts of the heart. Thus, depending on the location of the current injection electrodes and the site of the voltage sensing electrodes, the response will be different.

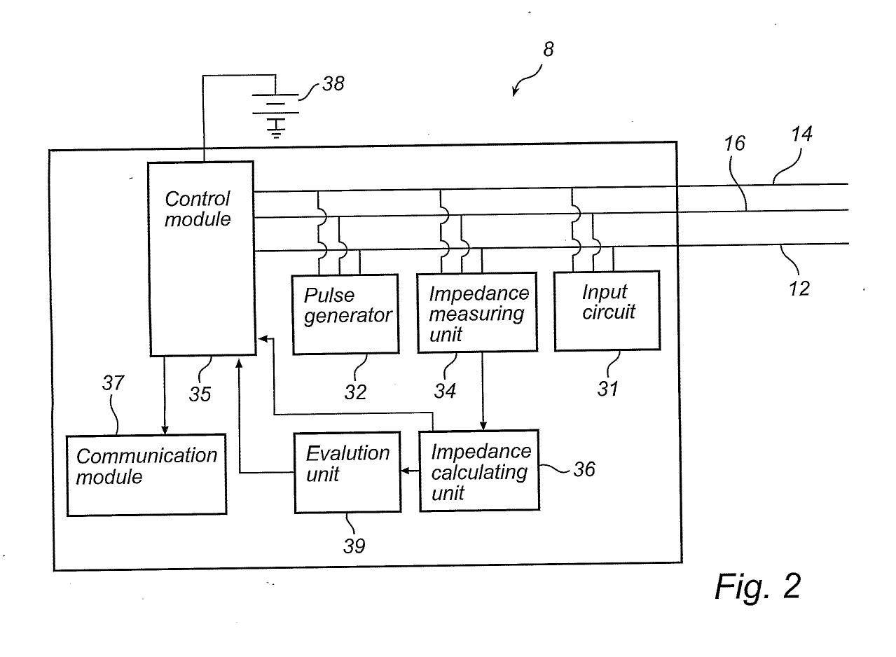

FIG. 2 is a block diagram illustrating the constituent components of an IMD 8 in accordance with the general principle of the present invention. In the following, a number of different embodiments of the present invention will be discussed and similar or like part, components, modules, or circuits through the different embodiments will only be described with reference to FIG. 2. Hence, in the description of the embodiments, the description of the similar or like part, components, modules, or circuits through the different embodiments will be omitted.

According to this embodiment, the IMD 8 is a pacemaker having a microprocessor based architecture. The leads 12 and 14 are connectable to the IMD 8 and comprises, as have been illustrated in FIG. 1, one or more electrodes, such a coils, tip electrodes or ring electrodes. These electrodes are arranged to, inter alia, transmit pacing pulses for causing depolarization of cardiac tissue adjacent to the electrode(-s) generated by a pace pulse generator 32 under influence of a control module or microcontroller 35. The rate of the heart 5 is controlled by software-implemented algorithms stored within a microcomputer circuit of the control module 35. The microcomputer circuit may include a microprocessor, a system clock circuit and memory circuits including random access memory (RAM) and read-only memory (ROM). The microcomputer circuit may further include logic and timing circuitry, state machine circuitry, and I/O circuitry. Typically, the control module 35 includes the ability to process or monitor input signals (data) from an input circuit 31 as controlled by a program code stored in a designated block of memory. The details and design of the control module 35 are not critical to the present invention. Rather, any suitable control module or microcontroller 35 may be used that carries out the functions described herein. The use of micro-processor-based control circuits for performing timing and data analysis functions are well known in the art.

Detected signals from the patient's heart 5, e.g. signals indicative of natural and stimulated contractions of the heart 5, are processed in an input circuit 31 and are forwarded to the microprocessor of the control module 35 for use in logic timing determination in known manner. The input circuit 31 may include, for example, an EGM amplifier for amplifying obtained cardiac electrogram signals.

IMD 8 comprises a communication unit 37 including an antenna (not shown), for example, a telemetry unit for uplink/downlink telemetry or RF transceiver adapted for bi-directional communication with external devices in, for example, the MICS band or ISM band.

Electrical components shown in FIG. 2 are powered by an appropriate implantable battery power source 38 in accordance with common practice in the art. For the sake of clarity, the coupling of battery power to the various components of the IMD 8 is not shown in the figures.

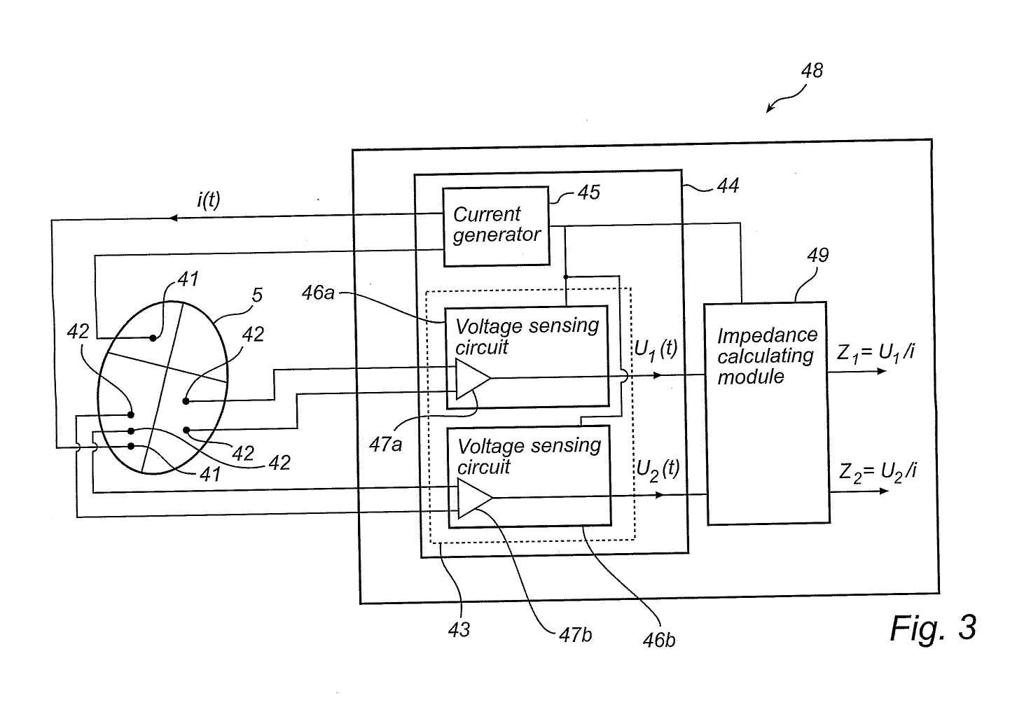

Furthermore, the IMD 8 comprises an impedance measuring unit 34 including a current generating device (see FIG. 3-5) adapted to generate a current and apply the current between two electrodes of the medical leads 12, 14, and/or 16, which current generating device 42 will be described in more detail below. The impedance measuring unit 34 further includes a voltage sensing device (see FIG. 3-5) including a plurality of voltage sensing circuits arranged in parallel, which voltage sensing device will be described in more detail below. Each voltage sensing circuit is connected to at least one electrode of the medical leads 12, 14 and/or 16 and is adapted to sense a voltage resulting from the applied current at the at least one electrode, wherein a voltage resulting from the applied current can be sensed by each voltage sensing circuit substantially simultaneously.

An impedance calculating module 36 is adapted to calculate impedance values, wherein each impedance value is based on the applied current and the sensed resulting voltage at a voltage sensing circuit. Hence, a set of impedance values can be produced at each impedance measurement session, for example, one impedance value for each voltage sensing circuit. Furthermore, the impedance calculating module 36 may be adapted to calculate average impedance values. For example, an average impedance value may be calculated for voltages measured with similarly oriented electrodes configurations such as RV tip electrode 22 and LV ring electrode 23, and RV ring electrode 24 and LV tip electrode 21, respectively. The voltage for these two configurations will look similar and will be affected by, for example, volume changes, in a similar manner. By averaging these signals, the signal to noise ratio will be reduced and thus the accuracy will be improved.

Moreover, the IMD 8 comprises an evaluation unit 39 adapted to evaluate the impedance values. In one embodiment, the evaluation unit 39 is an ischemia detector adapted to evaluate the impedance values, i.e. the obtained impedance matrix or pattern, including comparing the impedance values with a reference impedance matrix or pattern to detect changes in the impedance values being consistent with an ischemia and to determine a location of the ischemia. The reference impedance pattern may be created by the ischemia detector 39 by at least one impedance measurement obtained during a reference measurement session. For example, a patient specific reference impedance pattern can be created at a visit at the hospital or health-care institution a period of time after the implantation or at later visit.

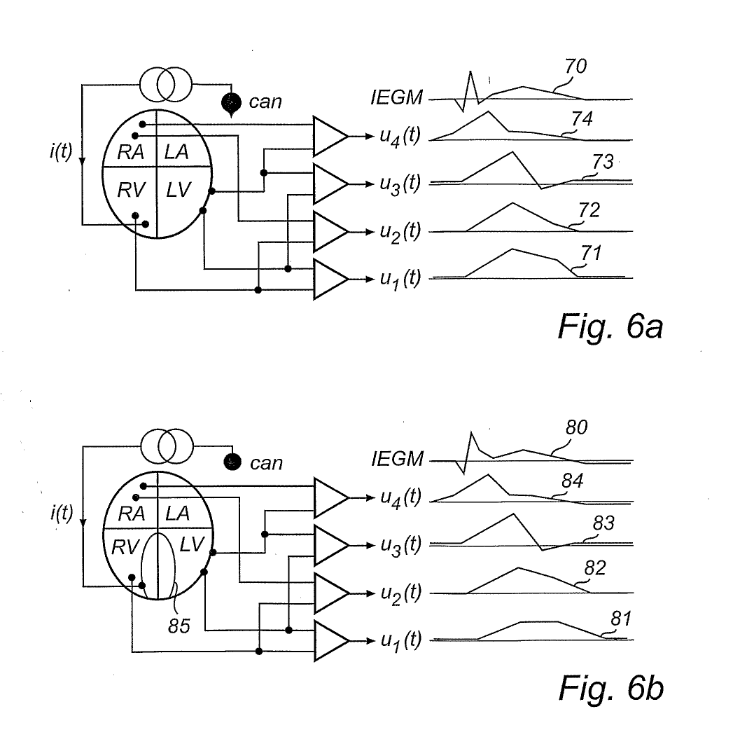

Hence, the ischemia detector 39 is adapted to evaluate the impedance values to detect changes in the impedance values being consistent with an ischemia and to detect a location of the ischemia. In particular, the impedance values are compared with a reference impedance matrix or pattern. If a part or a region of a heart is subjected to ischemia, this part or region will behave differently compared to the behaviour before the onset of the ischemia or in comparison to other non-ischemic parts or regions. Both the contraction and the relaxation of the myocytes will be slower during the ischemia. However, the hemodynamic behaviour can also change due to other circumstances such as, for example, a changed workload, change of body posture, medication, preload and afterload. Under such circumstances the myocardium will be affected globally and not regionally as in the case of an ischemic event. Therefore, the specificity of the ischemia detection can be improved by measuring the beat-to-beat response at several, different locations of the heart simultaneously. If the impedance values are gathered simultaneously during the same contraction at several areas of the heart (i.e. by means of several measurement vectors) and a change is observed in only a limited part of the heart (i.e. in one or only in few impedance values), it is therefore a high probability that an ischemic event is identified. Further, the location can also be identified since each impedance value is associated with a specific measurement vector, which in turn, measures the impedance in an area or a region of the heart. Thus, by localizing which vector(-s) that differ from a baseline (a reference) or from the other vectors an indication of which artery is subjected to coronary insufficiency or ischemia. On the other hand, if the change is seen in all impedance values (i.e. in all measurement vectors) it is more likely a global hemodynamic or metabolic change that not is a result of a local occlusion of a coronary artery. In FIGS. 6a and 6b, the impact on the impedance waveforms obtained by means of the different measurement vectors caused by an ischemic event is illustrated. In this example, the impedance is measured by means of four vectors. The voltages resulting from the injected current i(t) are u₁(t), u₂(t), u₃(t), and u₄(t). The right part of FIG. 6a illustrates the IEGM 70 and the impedance waveforms 71, 72, 73, and 74 measured during normal conditions. In FIG. 6b, an ischemic event 86 has occurred in the septum area or the region between the left ventricle LV and the right ventricle RV. The right part of FIG. 6b illustrates the IEGM 80 and the impedance waveforms 81, 82, 83, and 84 measured during this ischemic episode. As can be seen, the impedance waveforms obtained by means of the voltages u₁(t) and u₂(t) measured by the vectors measuring over the ischemic region is changed in comparison to the corresponding impedance waveforms obtained during normal conditions. Accordingly, by comparing obtained impedance waveforms to reference or baseline waveforms, it is possible to identify the onset of an ischemic event and the location of the ischemia. In FIGS. 6a and 6b, the impedance waveforms have been illustrated schematically and it is interesting to study, for example, the resistivity (the real part) and/or the reactance (the imaginary part) of the impedance and/or the phase angle and/or the time derivative of, for example, the resistivity or reactance, as well as the admittance in connection with ischemia detection. As can be seen, an ischemic event is consistent with a decrease in the amplitude of the impedance values and an extension of the duration of the impedance waveform, compare the waveforms 71 and 72 (normal situation) and the waveforms 81 and 82 (ischemia).

Furthermore, it also conceivable to study or analyse the resistivity and/or the reactance of the impedance and/or the phase angle, as well as the time derivative of any one, some or all of these parameters or admittance at different frequencies to detect an ischemic event and the location for the event as described in, for example, WO 2008/105692 by the same applicant entitled “Medical device for detecting ischemia and a method for such a device”, which hereby is incorporated in its entirety or “Myocardial Electrical Impedance Mapping of Ischemic Sheep Hearts and Healing Aneurysms”, Fallert A. Michael, et al., Circulation, Vol. 78, No. 1, January 1993.

The evaluation unit 39 may also determine the synchronicity between the chambers using impedance in the atrium measured simultaneously as the impedance in the ventricle is measured. The AV synchronicity is important in, for example, determining whether a detected fast ventricular rhythm originates from the ventricle of from the atrium.

Furthermore, the evaluation unit 39 may perform an optimization of the device parameter settings. For example, typical parameters that can be optimized are the AV delay and the W delay. Heart failure patients are sensitive to the excessively high stimulation rate at e.g. increased activity. Over-pacing and also too low pacing rate results in abnormal contraction patterns which can be detected by the simultaneous multisite impedance measurements. Analyses of the relation between the detector signals at different parameter settings give information about the change of cardiac contraction pattern, which can be of great value during, for example, continuous optimization of the heart stimulation. The optimal stimulation parameter setting can be identified and determined using echo or other external equipment, for example, at a set-up session at the health-care facility. An impedance pattern measured at a specific parameter setting or specific settings are recorded and stored as reference values. Thereafter, during the daily operation, the stimulation parameter settings may be continuously adjusted to obtain the same or a similar impedance pattern as the reference pattern.

In addition, the evaluation unit 39 may detect sudden ischemia or progression of existent ischemia. By using the impedance pattern obtained by the simultaneous multisite measurements of the impedance, i.e. the tissue response from different areas of the heart muscle, together with timing information, which can be provided with IEGM synchronized with the impedance measurements, and pacing events, it is possible to detect changes and mechanical contraction as well as the location where the change is observed. Hence, it is possible to alarm for sudden ischemic events and indicate the location of the ischemic event. It is also conceivable to initiate preventive therapy to avoid arrhythmic complications.

In FIG. 3 an embodiment of an implantable medical device according to the present invention including an impedance measuring unit and an impedance calculating module is schematically shown and will be discussed below. The IMD 48 shown is simplified and only parts and elements of the discussed embodiment of the present invention are shown in FIG. 3 and other parts and elements have been omitted.

The impedance measuring unit 44 includes a current generating device 45 connectable to electrodes 41 (for example a RA tip electrode and a RV ring electrode) within the heart 5. The current generating device 45 is adapted to generate a current i(t). Further, the impedance measuring unit 44 includes a voltage sensing device 43 comprising, in this illustrated embodiment, a first voltage sensing circuit and a second voltage sensing circuit 46a and 46b. Each voltage sensing circuit 46a and 46b comprises a differential amplifier 47a and 47b, respectively, and may further comprise additional circuits including, for example, input amplifiers (not shown), and low pass filters (not shown). Respective voltage sensing circuit 46a and 46b is connected to an electrode pair 42 (for example a RV tip electrode and a LV ring electrode, and a RV coil electrode and a LV tip electrode, respectively). A resulting voltage u₁(t) and u₂(t), respectively, is produced by the voltage sensing circuits 46a and 46b.

An impedance calculating module 49 is adapted to calculate impedance values based on the produced voltage values, and, in this illustrated embodiment, two impedance values are calculated:

Z₁=u₁/i

Z₂=u₂/i

As described above, the impedance values may be complex impedance values including the resistance and/or the reactance as well as the phase angle of the impedance. In such a case, an impedance pattern of the resistance and/or the reactance, which may be a conductance or an inductance, can be created. Further, an impedance pattern of the phase angle or the admittance can be created.

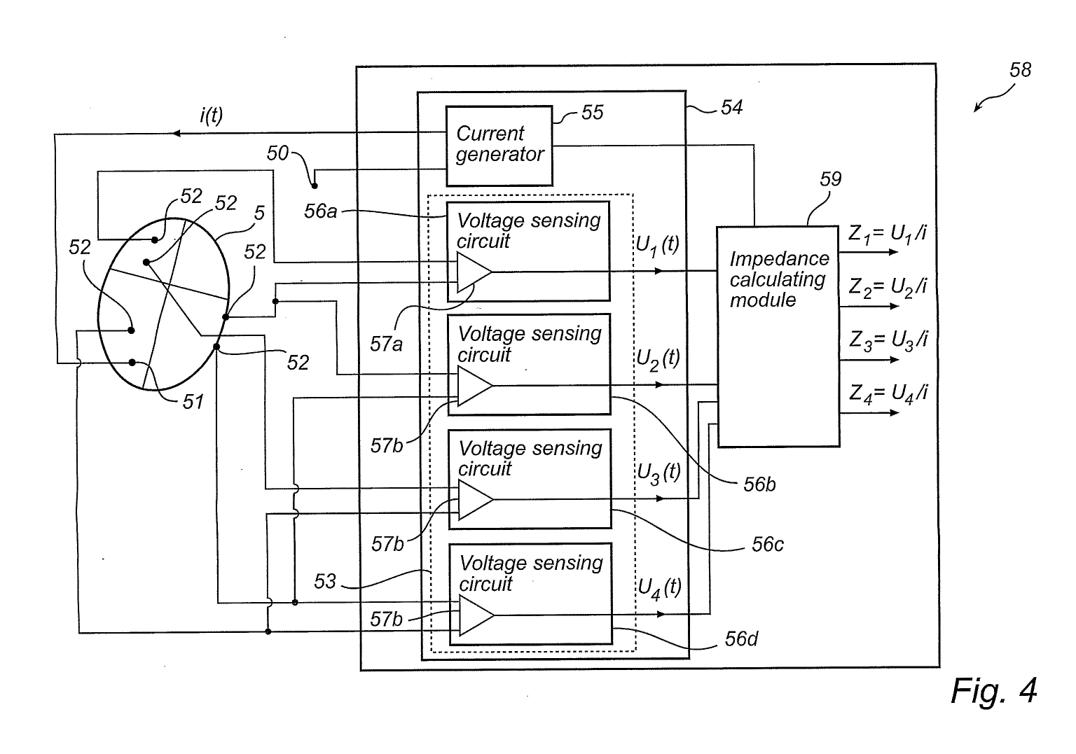

Turning now to FIG. 4, another embodiment of an implantable medical device according to the present invention including an impedance measuring unit and an impedance calculating module will be discussed. The IMD 58 shown is simplified and only parts and elements of the discussed embodiment of the present invention are shown in FIG. 4 and other parts and elements have been omitted.

The impedance measuring unit 54 includes a current generating device 55 connectable to electrode 50 (the can or housing) and electrode 51 (for example a RV tip electrode) within the heart. The current generating device 55 is adapted to generate a current i(t). Further, the impedance measuring unit 54 includes a voltage sensing device 53 comprising, in this illustrated embodiment, a first voltage sensing circuit, a second voltage sensing circuit, a third voltage sensing circuit, and a fourth voltage sensing circuit 56a, 56b, 56c, and 56d. Each voltage sensing circuit 56a-56d, respectively, comprises a differential amplifier 57a-57d, respectively, and may further comprise additional circuits including, for example, input amplifiers (not shown), and low pass filters (not shown). Respective voltage sensing circuit 56a-56d is connected to an electrode pair 52, for example a RA ring electrode and a LV ring electrode, located in e.g. epicardium, a LV ring electrode and LV tip electrode, located in e.g. epicardium, a RA tip electrode and a RV ring electrode, and a LV tip electrode and a RV ring electrode, respectively. A resulting voltage uᵢ(t)-u₄(t), respectively, is produced by the voltage sensing circuits 56a-56d.

An impedance calculating module 59 is adapted to calculate impedance values based on the produced voltage values, and, in this illustrated embodiment, four impedance values are calculated:

Z₁=u₁/i

Z₂=u₂/i

Z₃=u₃/i

Z₄=u₄/i

As indicated above, the impedance values may be complex impedance values including the resistance and/or the reactance as well as the phase angle of the impedance. In such a case, an impedance pattern of the resistance and/or the reactance, which may be a conductance or an inductance, can be created. Further, an impedance pattern of the phase angle or the admittance can be created.

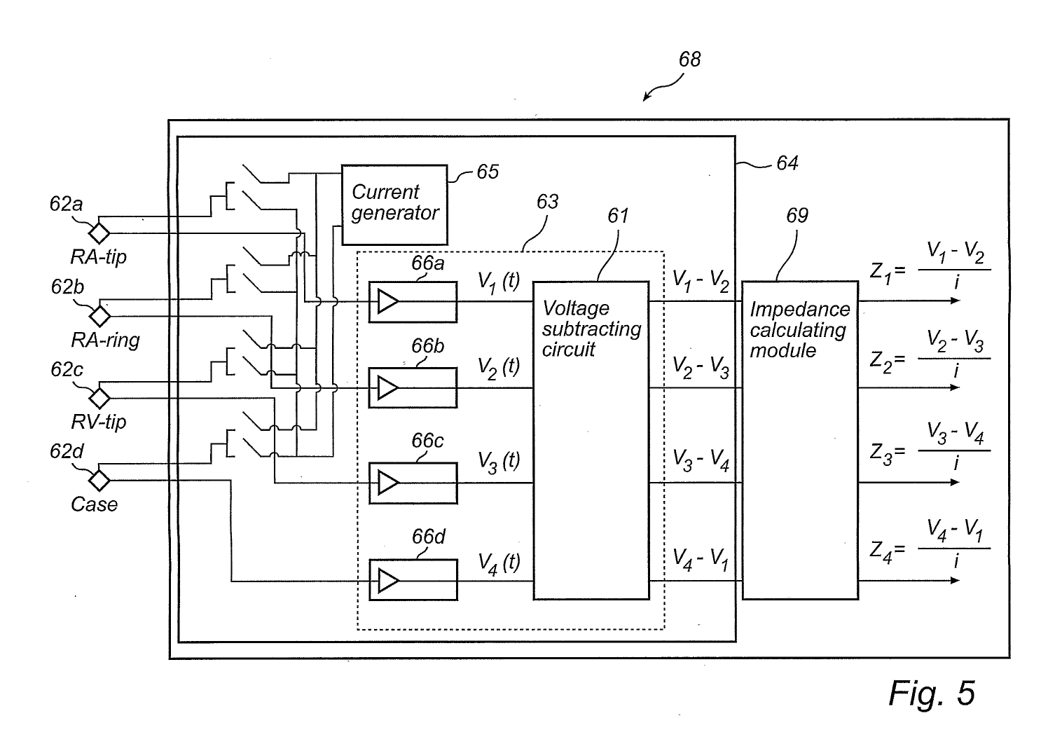

With reference to FIG. 5, a further embodiment of an implantable medical device according to the present invention including an impedance measuring unit and an impedance calculating module is schematically shown and will be discussed below. The IMD 68 shown is simplified and only parts and elements of the discussed embodiment of the present invention are shown in FIG. 5 and other parts and elements have been omitted.

The impedance measuring unit 64 includes a current generating device 55 connectable to electrodes, in this illustrated embodiment, an RA-tip electrode 62a, an RA-ring electrode 62b, an RV-tip electrode, and a case or can electrode 62d. The current generating device 65 is adapted to generate a current i(t). Further, the impedance measuring unit 64 includes a voltage sensing device 63 comprising, in this illustrated embodiment, a first voltage sensing circuit, a second voltage sensing circuit, a third voltage sensing circuit, and a fourth voltage sensing circuit 66a, 66b, 66c, and 66d. Respective voltage sensing circuit 66a-66d is connected to the RA-tip electrode 62a, the RA-ring electrode 62b, the RV-tip electrode, and the case or can electrode 62d. A resulting potential v₁−v₄, respectively, is produced by the voltage sensing circuits 66a-66d. Preferably, the respective output potential v₁−v₄ is referred to the ground potential. A voltage subtraction circuit 61 is adapted to calculate resulting voltages by subtracting two potentials, vₙ and vₘ, from each other.

An impedance calculating module 69 is adapted to calculate impedance values based on the produced voltage values, and, in this illustrated embodiment, four impedance values are calculated:

Z₁=(v₁−v₂)/i

Z₂=(v₂−v₃)/i

Z₃=(v₃−v₄)/i

Z₄=(v₄−v₁)/i

Although an exemplary embodiment of the present invention has been shown and described, it will be apparent to-those having ordinary skill in the art that a number of changes, modifications, or alterations to the inventions as described herein may be made. Thus, it is to be understood that the above description of the invention and the accompanying drawings is to be regarded as a non-limiting.

1. An implantable medical device connectable to at least one medical lead including a plurality of electrodes for contact with tissue of a heart of a patient, said device comprising:
an impedance measuring unit being connectable to a plurality of electrode configurations, said impedance measuring unit including
a current generating device adapted to generate a current and apply said current between two electrodes of a current injecting electrode configuration of said electrode configurations; and
a voltage sensing device including a plurality of parallelly arranged voltage sensing circuits, each voltage sensing circuit being connectable to a specific voltage sensing electrode configuration of said electrode configurations and being arranged to sense a voltage over said voltage sensing electrode configuration resulting from the applied current, wherein the voltage sensing circuits are capable of sensing the resulting voltages simultaneously; and
an impedance calculating module adapted to calculate a plurality of impedance values, each impedance value being based on the applied current and a resulting voltage.

2. The implantable medical device according to any one of preceding claims, further comprising an ischemia detector adapted to evaluate said calculated impedance values including comparing said impedance values with reference impedance values to detect changes in said calculated impedance values being consistent with an ischemia and to determine a location of said ischemia based on said detected changes.

3. The implantable medical device according to claim 2, wherein the ischemia detector is adapted to determine a location of said ischemia by identifying at least one impedance value being consistent with an ischemia, determining the at least one electrode configuration by which the respective at least one impedance value being consistent with ischemia was obtained, and determining the location of the ischemia to a region over which the at least one electrode configuration measures.

4. The implantable medical device according to claim 2 or 3, wherein a change of impedance values being consistent with an ischemia comprises a decrease of impedance amplitude values and/or an extension of a duration of an impedance waveform.

5. The implantable medical device according to claim 1, 2, or 3, wherein at least one of said voltage sensing circuits comprises a differential amplifier adapted to produce a differential voltage signal being proportional to a voltage difference between the electrodes of an electrode configuration connected to said voltage sensing circuit, and wherein an output voltage signal delivered to said impedance calculating module for impedance calculation is proportional to said voltage differential signal.

6. The implantable medical device according to any one of preceding claims 1-4, wherein said voltage sensing device comprises a voltage subtracting circuit being adapted to subtract a first voltage signal produced by a first voltage sensing circuit from a second voltage signal produced by a second voltage sensing circuit to determine a resulting voltage difference signal, wherein said voltage difference signal is delivered to said impedance calculation module for impedance calculation.

7. The implantable medical device according to claim 5, wherein each voltage sensing circuit is adapted to produce an output voltage signal with reference to a reference voltage and wherein said voltage subtracting circuit is adapted to determine said resulting voltage difference based on said output voltage signals.

8. The implantable medical device according to claim 1-6, wherein said impedance calculating module is adapted to calculate average impedance values using impedance values calculated by resulting voltages from predetermined electrode configurations.

9. A method for an implantable medical device connectable to at least one medical lead including a plurality of electrodes for contact with tissue of a heart of a patient and including an impedance measuring unit being connectable to a plurality of electrode configurations, said method comprising:
generating a current and apply said current between two electrodes of a current injecting electrode configuration of said electrode configurations;
sensing voltages at a plurality of parallelly arranged voltage sensing circuits, each voltage being sensed over specific voltage sensing electrode configuration resulting from the applied current, wherein the resulting voltages are sensed simultaneously; and
calculating a plurality of impedance values, each impedance value being based on the applied current and a resulting voltage.

10. The method according to claim 8, further comprising evaluating said calculated impedance values including comparing said impedance values with reference impedance values to detect changes in said calculated impedance values being consistent with an ischemia and determining a location of said ischemia.

11. The method according to claim 9, further comprising determining a location of said ischemia by identifying at least one impedance value being consistent with an ischemia, determining the at least one electrode configuration by which the respective at least one impedance value being consistent with ischemia was obtained, and determining the location of the ischemia to a region over which the at least one electrode configuration measures.

12. The implantable medical device according to claim 11 or 12, wherein a change of impedance values being consistent with an ischemia comprises a decrease of impedance amplitude values and/or an extension of a duration of an impedance waveform.
